(19) United States
(12) Patent Application Publication
(10) Pub. No.: US 2022/0389377 A1
(43) Pub. Date: Dec. 8, 2022

(54) TREATMENT OF MEDICAL CONDITIONS BY STEM CELL TRANSPLANTS AND STEM CELL ACTIVATION

(71) Applicant: Vitro Biopharma, Inc., Golden, CO (US)

(72) Inventors: James R. MUSICK, Golden, CO (US); Tiana STATES, Wheat Ridge, CO (US); Raymond BUNCH, Golden, CO (US)

(21) Appl. No.: 17/819,886

(22) Filed: Aug. 15, 2022

Related U.S. Application Data
(63) Continuation of application No. 17/239,513, filed on Apr. 23, 2021, now abandoned.
Application No. 17/239,513 is a continuation of application No. PCT/IB2022/053824, filed on Apr. 25, 2022.

(51) Int. Cl. C12N 5/074; A61K 38/00; A61K 35/28; A61K 35/30; A61K 38/55

(52) U.S. Cl. CPC C12N 5/0607; A61K 38/005; A61K 35/28; A61K 35/30; A61K 38/55; C12N 2500/76; C12N 2501/999; C12N 2500/05; C12N 2500/30

(57) ABSTRACT

A method of treating medical conditions by stem cell therapy. Stem cells are transplanted into and onto a human or animal patient in need, Then stem cell proliferation is increased, activating migration to sites of inflammation and epigenetic effects, regenerating damaged tissues through inhibition of apoptosis, inhibition of inflammation and oxidative stress, and inducing angiogenesis and stem cell differentiation into various cells by administering substances that induce GSK-3 beta inhibition and HDAC inhibition.

### CROSS-REFERENCE TO RELATED APPLICATION

This application is a Continuation of U.S. patent application Ser. No. 17/239,513, entitled “TREATMENT OF MEDICAL CONDITIONS BY STEM CELL TRANSPLANTS AND STEM CELL ACTIVATION”, filed Apr. 23, 2021, and a Continuation of International Patent Application No. PCT/IB2022/053824, entitled “TREATMENT OF MEDICAL CONDITIONS BY STEM CELL TRANSPLANTS AND STEM CELL ACTIVATION”, filed Apr. 25, 2022. The contents of all of the above-noted applications are incorporated herein by reference as if set forth in full and priority to these applications is claimed to the full extent allowable under U.S. law and regulations.

### FIELD OF THE INVENTION

The present invention is related to treatment of various medical conditions and, more particularly, to treatment of various medical conditions by transplanting mesenchymal stem cells into a patient in need and then activating stem cells in the patient through specific nutraceuticals and combinations that induce proliferation, migration, and epigenetic reprogramming of stem cells thus enhancing therapeutic benefits.

### BACKGROUND OF THE INVENTION

Stem cell therapy is a common procedure for the treatment of blood disorders including leukemia, lymphoma and auto-immune conditions using transplantation of hematopoietic stem cells (HSCs). Other types of adult stem cells are now being transplanted to treat other conditions including skeletal muscular disorders, cardiovascular diseases, and cardiomyopathy. While activation of endogenous stem cells has been used to increase proliferation and differentiation of hematopoietic stem cells for several years, such activation of other adult stem cells including mesenclhv gal stein cells (MSCs), satellite cells, and neural stem cells (NSCs) has not yet reached routine clinical practice.

Adult mesenchymal stem cells are particularly relevant to the present invention. These cells were initially described by Arnold Caplan (Caplan, Al and Bruder, S P, Trends Mol Med 7: 259-264, 2001). MSCs may be derived from various tissues including bone marrow, adipose-tissue, dental pulp, umbilical cord, amniotic fluid and membranes, placenta, and other sources well known to those skilled in the art. Comparative studies suggest differences in potency, differentiation capacity, growth rates and other cellular characteristics depending on the tissue source used to procure MSCs. This may be related to several factors, including medical status of the donor and environmental factors specific to the stem cell niche.

MSCs derived from bone marrow and adipose tissue have been subject to numerous clinical trials that provide preliminary evidence of safety and efficacy. A classic feature of MSCs is trilineage differentiation into adipocytes, chondrocytes, and osteoblasts, although multiple other cellular lineages may be derived from MSCs, including neural, kidney, and cardiac cells. Hence, MSCs have been extensively studied in skeletal muscular conditions such as osteoarthritis (OA). Several studies support safety and efficacy by intra-articular injections into knees, hips, and shoulder joints of OA patients. In addition, several other conditions may be treated by MSC transplants or transplants of progenitor cells derived from MSCs including stroke, myocardial infarct, and congestive heart failure.

Stem cell-based regenerative medicine is revolutionizing the treatment of medical conditions by treating underlying causes of injury and disease through rejuvenation and replacement of dead or injured cells and tissues. Thus, for example, the standard of care for treating osteoarthritis of joints is anti-inflammatory and pain management followed by prosthetic joint replacement. However, several clinical research groups (Lijima, H, et al, NPJ Regenerative Medicine (2018) 3:15) are providing clinical data showing that a single injection of MSCs into an arthritic joint relives pain, restores joint function and induces the regeneration of cartilage thus obviating the need for a joint replacement. Additional clinical and pre-clinical studies show regenerative effects of MSCs and neural stem cells in various neurological conditions including stroke, Parkinson's disease and other conditions characterized by death of particular types of neurons. Since these changes last for considerable periods of time, the stem cell regenerative effects are apparently curative rather than treating just symptoms as is common with the current standards of care.

### DESCRIPTION OF RELATED ART

A detailed study published in 2013 by scientists at the National Institutes of Health (NIH) showed reduction in lesion volume, diminution of blood brain barrier disruption, reduced neuronal hippocampal degeneration and enhanced recovery of motor coordination in a mouse model of traumatic brain injury (TBI) by combined treatment with lithium and valproic acid, sodium valproate, and sodium divalproex (Li-VPA). Yu, F., et al., J. Neurosurg. 119: 766-773, 2013.

Examples 5 and 6 in the present application show that Li-VPA activates MSCs and NSCs in a dose dependent manner suggesting that the mechanism for TBI treatment involves activation of endogenous stem cells. TBI is caused by a bump, blow, or jolt to the head via penetration of the brain or closed head injury. Some 5.3 million Americans (i.e., 2% of the United States population) currently live with disabilities resulting from TBI.

### SUMMARY OF THE INVENTION

The present invention teaches new methods and provides compositions related to stem cell therapy that includes transplantation of stem cells followed by activation of both exogenous and endogenous stem cells to regenerate damaged tissues through various biological processes including inhibition of apoptosis, inflammation, oxidative stress and the induction of angiogenesis in addition to differentiation of stem cells into various cell types. The present invention arose from the discovery that specific nutraceutical formulations could reproduce the proliferation, migration and epigenetic effects of Li-VPA as shown in examples 3, 8 and 9, hereinbelow. An embodiment of the invention is the combination of specific nutraceutical compounds that induce stem cell activation either alone or in combination with a MSC transplant into a patient in need. In addition, curcumin and specific formulations of curcumin and quercetin, curcumin and resveratrol are also embodied for the activation of stem cells and treatment of medical conditions.

For example, following treatment with stem cell activating agents, the stem cells may then be transplanted into the patient for various uses such as treatment of spinal cord injury, stroke, Alzheimer's disease, Parkinson's disease, and other conditions amenable to therapy by stem cell transplantation. Biological fluid analysis also includes pharmacodynamic endpoints of therapies such as measurement of histone acetylation, GSK3-β inhibition, and other relevant biomarkers. The methods and compositions of the present invention are intended for treatment of various medical conditions, including but not limited to ARDS secondary to viral infection, osteoarthritis, tendonitis, herniated disc, ligament damage, acute and chronic kidney disease and injury, Parkinson's disease, traumatic brain injury, Alzheimer's disease, amyotrophic lateral sclerosis, spinal cord injury, other skeletal muscular disorders, Lupus erythematosus, multiple sclerosis, Crohn's disease, cardiovascular disease, Graft-versus host disease, liver dysfunction or diseases, Type 1 diabetes, Type 2 diabetes, myocardial ischemia, heart failure, coronary artery disease, and other disorders characterized by inflammation.

In accordance with the present invention, there is provided a method of treating medical conditions by stem cell therapy. Stem cells are transplanted into and onto a human or animal patient in need, Then stem cell proliferation is increased, activating migration to sites of inflammation and epigenetic effects, regenerating damaged tissues through inhibition of apoptosis, inhibition of inflammation and oxidative stress, and inducing angiogenesis and stem cell differentiation into various cells by administering substances that induce GSK-3 beta inhibition and HDAC inhibition.

### BRIEF DESCRIPTION OF THE DRAWINGS

The patent or application file contains at least one drawing executed in color. Copies of this patent or patent application publication with color drawings will be provided by the Office upon request and payment of the necessary fee.

A complete understanding of the present invention may be obtained by reference to the accompanying drawings, when considered in conjunction with the subsequent detailed description, in which:

FIG. 1 is a microphotograph depicting cell migration using fluorescent readout and cell tracker green as a fluorescent marker of human MSCs;

FIG. 2 is an image illustrating MSC migration as percent closure as a function of time during live-cell data acquisition at different dosages of Substance P;

FIG. 3 is a line graph representation illustrating migration of various human cell lines exposed to Substance P;

FIG. 4 is a line graph representation illustrating less than 1 μM curcumin induced migration of human MSCs and 1 to 10 μM curcumin blocked migration due to apparent toxicity;

FIG. 5 is a line graph representation illustrating migration of MSCs induced by exposure to lithium alone (green diamonds), VPA alone (black squares) and VPA in the presence of 200 μM lithium (red circles);

FIG. 6 is a line graph representation illustrating migration of MSCs, NSCs and colorectal CAFs induced by increasing VPA concentration in 200 μM lithium;

FIG. 7 is a line graph representation illustrating migration of human MSCs exposed to a combination of lithium and VPA with and without inhibition of MMP9 and CXCR4;

FIG. 8 is a line graph representation illustrating proliferation of different cell lines induced by increasing concentrations of FGF-b (FGF-2) after a 5 day exposure, FGF being the family of fibroblast growth factors;

FIG. 9 is a line graph representation illustrating proliferation of MSCs induced by lithium, VPA and VPA in 200 μM lithium after a 5 day exposure;

FIG. 10 is a line graph representation illustrating proliferation of MSCs and NSCs as a function of increasing VPA in 200 μM lithium after a 5 day exposure;

FIG. 11 is a bar graph representation illustrating qPCR used to measure target genes known to be subject to epigenetic regulation by HDAC inhibitors including VPA Oct 3/4, a pluripotency gene, Sirt-1, age-related gene and FGF-21, whose expression is related to VPA-Li synergy;

FIG. 12 is a bar graph representation illustrating cytokine levels in cell culture media exposed to MSCs for 24 days and determined by microarray analysis;

FIG. 13 shows the expression levels of β-actin, Oct 3/4, Sirtuin I, FGF21, CXCR4 and Hsp70 in human MSCs exposed various epigenetic agents as determined by qPCR;

FIG. 14 shows the activation of human MSC migration by various concentrations of quercetin in the presence of 500 nM curcumin and the inhibition of migration by 20 μM AMD3100 and 15 μM GM6001 which are specific inhibitors of CXCR4 and MMP9, respectively; and

FIG. 15 shows the activation of human MSC migration by various concentrations of resveratrol in the presence of 500 nM curcumin and the inhibition of migration by 20 μM AMD3100 and 15 μM GM6001 which are specific inhibitors of CXCR4 and MMP9, respectively.

### DETAILED DESCRIPTION OF THE INVENTION

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

Stem cells of the present invention are derived from a variety of tissues including adipose tissue, umbilical cord, placenta, amniotic fluid or membranes, umbilical cord blood, bone marrow, and other sources well known to those skilled in the art. The MSCs are extracted from tissue through a combination of microdissection followed by enzymatic or mechanical dissociation. Donor screening is used to eliminate those with pre-existing conditions that may impose a safety risk for use of stem cells in clinical applications. MSCs are purified by a variety of methods such as flow cytometry or selective adsorption to plastic. Expansion occurs by standard methods of cell culture, preferably performed under conditions of reduced oxygen (1% to 5%). Expansion of cells occurs by serial passage in cell culture using various methods of cellular dissociation from culture flasks/plates well known to those skilled in the art. MSCs are then characterized by a variety of methods to ensure authenticity by function including differentiation capacity, growth rate, ATP production, expression of indoleamine 2,3-dioxygenbase induced by γ-IFN and other MSC functional characteristics known to those skilled in the art. Also, phenotypic characterization is performed using flow cytometry to determine the absence or presence of specific cellular biomarkers. Karyotyping, DNA finger printing, and other well known methods are used to authenticate the species of origin and elucidate the genome of the MSC line. Adventitious viral agents are determined by specific PCR methods or broader in-vitro and in-vivo methods detecting known and unknown viruses. Detection of bacteria, fungi, and viruses is used to eliminate transmission of these agents during stem cell transplantation. Clinical use of MSCs requires rigorous adventitious agent testing by in-vitro cultures of well characterized cell lines, in-vivo testing in various animal species and testing of any animal-derived products used in the manufacturing process.

The MSC lines derived by the above methods are formulated by various methods including cryopreservation, viable liquid formulations that are suitable for administration into patients through various routes including both systemic and local applications. The MSCs may be genetically modified prior to clinical use and secretion products of stem cells including exosomes may be used instead of MSCs.

The MSCs are distributed to various administration sites in suitable, stable formulations to accommodate logistic requirements including temperature maintenance, and continuous monitoring of environmental parameters. Following recovery from cryopreservation by methods well known to those skilled in the art as needed, stem cells are administered to a patient in need by various routes of administration including, but not limited to, intravenous infusion, intra-articular injection, intra-spinal-disc injection, or other methods of systemic administration or local injection or implantation well known in the art, including combination with stabilizing agents such as extracellular materials, either natural or synthetic. Medical conditions suitable for the stem cell therapies embodied within the present invention include without limitation: ARDS secondary to viral infection, osteoarthritis, tendonitis, herniated disc, ligament damage, acute and chronic kidney disease and injury, Parkinson's disease, traumatic brain injury, Alzheimer's disease, amyotrophic lateral sclerosis, spinal cord injury, other skeletal muscular disorders, Lupus erythematosus, multiple sclerosis, Crohn's disease, cardiovascular disease, Graft-versus host disease, liver dysfunction or diseases, Type 1 diabetes, Type 2 diabetes, myocardial ischemia, heart failure, coronary artery disease, and other disorders characterized by inflammation.

Disclosed herein are methods for activating stem cells, including methods for inducing stem cell proliferation, migration and epigenetic reprogramming. A medical condition can be treated in a subject in need thereof, the steps comprising administering nutraceuticals and combinations thereof. As used herein, nutraceuticals are defined as naturally occurring compounds that possess particular biological activities including, without limitation, epigenetic modulation, induction of stem cell migration and/or proliferation.

Adult stem cells reside in various regions throughout the body of warm-blooded animals including bone marrow where both hematopoietic and mesenchymal stem cells reside, satellite cells within muscles, and various MSCs within other tissues (e.g., teeth pulp, endocrine glands, arteries as pericytes). Within the nervous system, NSCs are concentrated within the subventricular zone (SVZ) of the lateral ventricles, the dentate gyrus of the hippocampus, within the olfactory epithelium and diffusely within the frontal cortex.

The rostral migratory stream (RMS) is a CNS structure of continuous flow of neural progenitor cells, neural stem cells and transiently activated neural progenitor cells whereby a migratory stream of cells provides potential regeneration of the adult brain through cellular activation at the level of the SVZ in the lateral ventricles. The RMS is a critical component of brain development and also maintains activity within the adult brain. Activation of stem cells in the SVZ through increased proliferation, migration and epigenetic reprogramming is an embodiment of the present invention.

While cells migrating through the RMS are ultimately destined for the olfactory bulb, there are circumstances that promote migration of NSCs away from the RMS into other regions of the brain. Injury is known to induce migration of NSC and related progenitors to sites of injury and inflammation through chemical signaling involving chemokines ligands and their chemokine receptors. These include the Stromal-derived factor 1a (SDF1a, also known as CXCL12 chemokine ligand 12 and its receptor, CXCR4 chemokine receptor 4 and related signaling systems).

Injury includes sites of injury and inflammation characterized by primary pro-inflammatory cytokines including IL-1 and TNF, etc. that trigger secondary inflammatory chemokines including CXCL12 whether due to TBI, cell death from concussion, cellular degeneration due to neurodegenerative diseases, local regions of inflammation due to infection including meningitis, cell death induced by reduced blood flow/hemorrhage, etc. Other chemokines and factors are involved as well in the migratory process that ultimately results in the movement of neural stem cells into regions of cerebral injury. Additional factors are also known to induce ectopic migration to surrounding brain regions including changes in extracellular matrix proteins including neural cell adhesion molecule (NCAM) and tenascin-R. (Sun, W., et al. Anat Cell Biol 43:269-279, 2010.)

While hematopoietic stem cells (HSCs) are relatively active and responsible for production of 200 billion red blood cells daily, other niche regions of adult stem cells are typically quiescent and are activated by a specific stimulation, such as response to injury. Stem cell activation, as referred to herein, involves three separate, although not necessarily independent, biological processes: proliferation, migration and epigenetic reprogramming. Proliferation increases the number of stem cells through cell division, while migration is characterized by movement to specific targets mediated through chemokine/chemokine receptor systems within stem cells and the local environment. This migration of stem cells allows for various regenerative and repair processes to occur at sites remote from the actual stem cell niches within the body.

Epigenetic re-programming involves increased expression of specific genes involved with stem cell pluripotency (e.g., Oct 3/4—Octamer-binding transcription factor 3/4) and longevity (Sirtuin family including Sirtuin-1 or SIRT-1) and certain members of the fibroblast growth factor (FGF) family of proteins, including FGF-21. Epigenetic reprogramming is mediated through HDAC inhibition resulting in modulation of DNA methylation patterns yielding altered gene expression including increased or decreased expression of specific genes. Proliferation is induced by several molecular mechanisms that yield increased numbers of cells through the process of cell division. Other activating agents are also disclosed herein, including specific agents that provide potent and selective inhibition of GSK3-β (glycogen synthetase 3-β) and HDAC-I (histone deacetylase I).

Also, stem cell activation agents may be administered in a variety of different routes of drug delivery, including orally, by use of a nasal spray, intra-thecal injection, eye drops, dermal delivery systems, etc.

Another embodiment refers to concomitant treatments to both activate neural stem cells and to enhance the migration beyond the RMS to other brain regions. Neural stem cells are derived primarily from cell division in the SVZ of the lateral ventricle and then migrate through the RMS eventually differentiating into neurons within the olfactory bulb. Injury, cell degeneration, infection, etc. result in collateral migration to these sites from the RMS. This signaling is mediated through various factors elaborated at the injured site that promote tropic movement of NSCs away from the RMS. The RMS is composed of complex network of cells, vasculature and extracellular matrix molecules (ECMs) that usually limit cell movement to the RMS itself.

Specific ECM molecules especially NCAM and Tenascin R, play significant roles in the migration of NSCs within the RMS. NCAM is particularly important in controlling RMS cellular migration; it interacts with itself or other molecules to regulate cellular migration within the RMS.

The addition of homopolymers of sialic acid (polysialic acid or PSA) is a highly regulated post-translational modification of the neural cell adhesion molecule (NCAM). Two enzymes, the polysialyltransferases, ST8Sial I (ST8Sial) and ST8Sial V (ST8SiaV) are responsible for the biosynthesis of PSA. Since the removal of PSA from NCAM is known to allow diffuse migration of NSC and related cells from the RMS to surrounding CNS regions (Battista and Tutishauer, J. Neurosci 30:3995-4003, 2010) the present invention includes concomitant methods to activate neural stem cells within the brain and particularly in the SVZ of the lateral ventricles together with methods to modify and limit the restraints from ectopic migration by interference with the process of PSA addition to NCAM. For example and without limitation, this may include use of synthetic precursors including N-butyl mannosamine, small inhibiting RNA to ST8Sial, ST8SiaV, peptide mimetics that block addition of PSA to NCAM, and other enzymatic inhibitors and negative regulators of expression that result in blocking the addition of PSA to NCAM.

In another embodiment, the present invention includes compositions yielding desired levels of GSK3-β and HDAC-I inhibition. Thus, a single formulation of Li and PA is provided wherein the composition yields a concentration of Li ranging from 0.1 to 0.4 mM and VPA at 2.5 to 15 μg/ml with excipients and additives to achieve the desired local concentrations at NSC regions of the brain.

A further embodiment includes compositions comprising substances in addition to Li and VPA that have potent and selective ability to inhibit GSK3-β and HDAC-I. These compositions include, but are not limited to, curcumin as a GSK3-β inhibitor and Romidepsin (FK-228) as a potent and selective HDAC-I inhibitor. As shown in example 8, hereinbelow, curcumin also has epigenetic effects, probably through HDAC inhibition (Soflaei S S, et al, Curr Pharm Des. 2018; 24(2):123-129) resulting in increased expression of Oct 3/4, Sirt-1, FGF21, CXCR4 and Hsp70 that were comparable to the levels induced by VPA. Thus, the pleiotropic agent, curcumin, represents a single agent that is both a GSK3-β inhibitor and also inhibits HDAC.

An additional embodiment includes combinations of nutraceuticals to induce stem cell activation. More specifically, curcumin and quercetin combine to activate stem cell migration at preferred dosages of 500 nM curcumin and 500 nM quercetin (Example 9, hereinbelow).

A further embodiment includes the combination of curcumin and resveratrol at the preferred concentrations of 500 nM curcumin and 500 nM resveratrol (Example 9, hereinbelow).

Bioavailability of pharmacological and nutraceutical agents is an important component of therapy. Thus, liposomal formulations of curcumin and other formulations including piperine are used to maintain adequate bioavailability. The compositions of the present invention thus include various compounding processes well known to those skilled in the art to alter pharmacokinetic properties and combine appropriate dosages of GSK3-β inhibitor and HDAC-I inhibitors into single medications. Also, various administration routes may be utilized within the present invention to optimize drug delivery to CNS, Thus, for example but without limitation, activating drugs and NCAM modulating substances may be compounded together or alone into nasal sprays, eye drops, appropriate formulations for intrathecal delivery, dermal patches, etc. These formulations may involve the appropriate use of nano technology, liposomes; emulsions, ointments, etc. as are well known to those skilled in the art.

In another embodiment, the present invention involves the use of cell-based assays to provide optimal therapy to patients, including all warm-blooded animals. Cell-based assays include stem cells that the therapy is designed to activate in a suitable culture system to allow maintenance of appropriate environment of cell culture media, temperature, relative humidity, oxygen, and carbon dioxide level. In one embodiment the stem cells may be allogeneic and represent specific classes of adult stem cells such as hematopoietic, mesenchymal, neural, muscle, etc. Use of such cultures is ideal for studies of basic issues such as dosage and comparative studies. Another embodiment involves use of patient-specific stem cells, expansion of these cells and use of the cells in cell-based assays of a specific element. For example, human neural stem cells may be derived by biopsy of nasal epithelia tissues. (Girard, S D, et al., J. Vis Exp 54: e2762, 2011). Enzymatic digestion according to methods well known in the art may be used to disperse and purify cells followed by cell culture using established methods of NSC expansion resulting in patient-specific NSC cultures. Preferably, these cultures are maintained on laminin or fibronectin-coated (or equivalent) tissue culture plates or flasks in a medium containing the ROCK inhibitor, Y27632 at 10 to 40 μM final concentration and also containing DYRK (dual-specificity tyrosine-phosphorylation-regulated kinase) inhibitor, ID-8 at 0.5-10 μM final concentration.

These cultures provide numerous advantages, including allowing the determination of personalized dosage regimes, and the direct study of cellular responses including proliferation, migration and epigenetic reprogramming. In addition the cell culture media may be collected from said cultures and analyzed for biomarker content to determine baseline secretion levels and those resulting from stem cell activation using Li and VPA and other activating agents as well. These cell-based assays are also amenable to discovery and characterization of novel activating agents as well. It should be noted that stem cell activation does not necessary involve a chemical process but may also occur through the use of appropriate energy input into stem cells, such low-power laser activation, exposure to electrical/magnetic fields or light at specific intensities and frequencies.

Gene expression profiling also provides detailed analysis of the epigenetic reprogramming resulting from epigenetic modulation as by HDAC inhibition and the subsequent alterations in DNA methylation. This same approach may be extended to other patient-specific cellular samples besides NSCs derived from nasal epithelium biopsies including, without limitation, mononuclear cells contained in whole blood.

A further embodiment includes use of patient-specific stem cells for treatment of various conditions by stem cell transplantation. Such stem cells may be provided to the patient in various conditions including quiescent cultures of viable cells or as activated cells such as those exposed to Li-VPA, curcumin-quercetin or curcumin-resveratrol. It has been shown that such activation results in increased recovery of spinal cord injury when compared to non-activated cells. Such cells, either activated or not, may be used therapeutically through transplantation to treat other conditions amenable to stem cell therapy including, for example, macular degeneration, stroke, Alzheimer's disease, Parkinson's disease, etc. Also, a further extension of this embodiment includes use of differentiating agents to drive stem cells, which may be either allogeneic or autologous, into particular lineages. Thus, for example, patient NSCs may be differentiated into cholinergic or dopaminergic neurons for use in autologous stem cell therapy of Alzheimer's or Parkinson's disease, respectively. Other differentiated cell lineages and therapies are readily apparent to those skilled in the art and are not intended to be limited from this embodiment.

Analysis of stem cell activation according to the present invention also includes neuro-imaging methods to assess NSC status within patients. While many imaging procedures provide information on multi-cellular structures, other methods well known in the art allow imaging at a cellular level. Pyrimidines are selectively taken up by proliferating cells and a preferred, without limiting, method of NSC proliferation imaging of the present invention is by positron-emission tomography of F-labeled 3′-deoxy-3′-fluorothymidine at known anatomical NSC niches including the SVZ of the lateral ventricles. Other embodiments of this method of direct imaging of stem cell proliferation are readily apparent to those skilled in the art.

### Examples

A series of assays quantified various functional aspects of stem cell activation including proliferation, migration, epigenetic reprogramming and stem cell secretome analysis. These assays were validated through comparison to prior results. Results show that Li and VPA enhance proliferation and migration of MSCs and NSCs in a dose-dependent manner. Cell migration by VPA was inhibited by blockage of CXCR4 and Li-induced cell migration was inhibited by blockage of MMMP-9, suggesting their involvement in the mechanism of stem cell activation. Additional molecular mechanisms are possible. The data suggest a role for epigenetic modulation in stem cell activation.

Example 1: Expansion of cell lines for use in cell-based assays: Native human umbilical cord-derived MSCs (Vitro Biopharma Cat. No. SC00A1), human pancreatic fibroblasts (Vitro Biopharma, Cat. No. SC00A5), colorectal cancer-associated fibroblasts (Vitro Biopharma, Cat. No. CAF05), and pancreatic stellate cancer-associated fibroblasts (Vitro Biopharma, Cat. No. CAF08) were plated at 7500 cells/cm² and grown to 90% confluency in T-25 tissue culture (TC) flasks (BD Falcon, Cat. No. 353108) in MSC-Gro™ low serum, complete medium (Vitro Biopharma Cat. No. SCO0B1). Neural stem cells (Vitro Biopharma, Cat. No. SC00A1-NSC), were similarly cultured except that neural MSC-Gro™ medium (Vitro Biopharma, Cat. No. NSCB1) and laminin-coated T-25 flasks (Corning BioCoat, Cat. No. 354533) were used. Cultures were maintained in a humidified chamber equilibrated with 5% CO₂, 1% O₂, balance N₂ at 37° C. Cells were detached using Accutase™ (Innovative Cell Technologies Inc., Cat No. AT-104) and collected by centrifugation (450×g) for 7 minute. Following aspiration, the cell pellet was resuspended in 1 mL PBS and cells were counted using a Beckerman-Coulter Z2 particle counter (range 10 μm-30 μm).

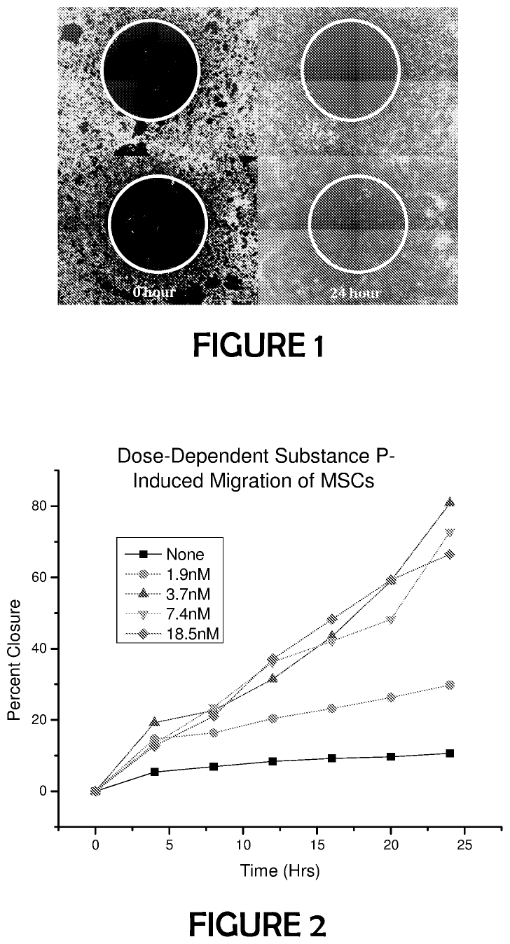

Example 2: Stem Cell Migration Assay: In order to investigate the effects of drugs and nutraceuticals on human stem cell migration, a stem cell-based assay of migration was developed and validated. FIG. 1 shows the basic components of the assay and shows the effects of substance P, a well known inducing agent of cell migration. FIG. 1 is a microphotograph depicting cell migration using fluorescent readout and cell tracker green as a fluorescent marker of human UC-MSCs. These cell images show fluorescent human MSCs (green) at the beginning of the assay (left panel) of the control vs. activating agent (Substance P at 3.7 nM)) and 24 hours later (right panel). MSCs migrated to the cell-free center of the well and also filled open areas in other regions of the culture as a result of Substance P exposure (lower right panel) but did not similarly migrate in its absence (upper right panel). The EC₅₀ was determined as a measure of effectiveness of activating agents. Initial kinetic data allowed determination of the optimal assay parameters for further experiments. An initial analysis of the cell free zone using Image J software was used to screen concentrations of activating agents for dose-response determination in further experiments. It was determined that MSCs plated at 25,000/well, incubated for 24 hours and then exposed to appropriate activator concentrations gave optimal results.

FIG. 1 shows the results of testing the effect of 3.7 nM Substance P on the migration of human UC-MSCs. In the absence of Substance P (upper panels), no migration was detected into the cell-free zone created by culturing cells in the presence of an occluding plug that prevented cell attachment in the center of the well during 24 hours of culture. This was a consistently observed result under control conditions (i.e., no activator). On the other hand, 3.7 nM Substance P-induced MSC migration into the cell-free center of the well and increased fluorescence within the original, cell containing region of the well (FIG. 1, lower two panels). Analysis of fluorescence within the cell-free region at the center of the well was used as a quantitative measure of cell migration as described below.

FIG. 2 is a line graph representation illustrating percent closure as a function of time during live-cell data acquisition at different dosages of Substance P. FIG. 2 shows the dose-response of Substance P-induced migration of CB-MSCs. Percent closure is shown under control conditions (no Substance P) and with increasing concentrations of Substance P as a function of time during the 24-hour period of live cell analysis. No migration is seen without Substance P, while migration increased in a dose-dependent manner by exposure to Substance P from 0 to 18.5 nM. Migration exhibited saturation at higher Substance P concentrations.

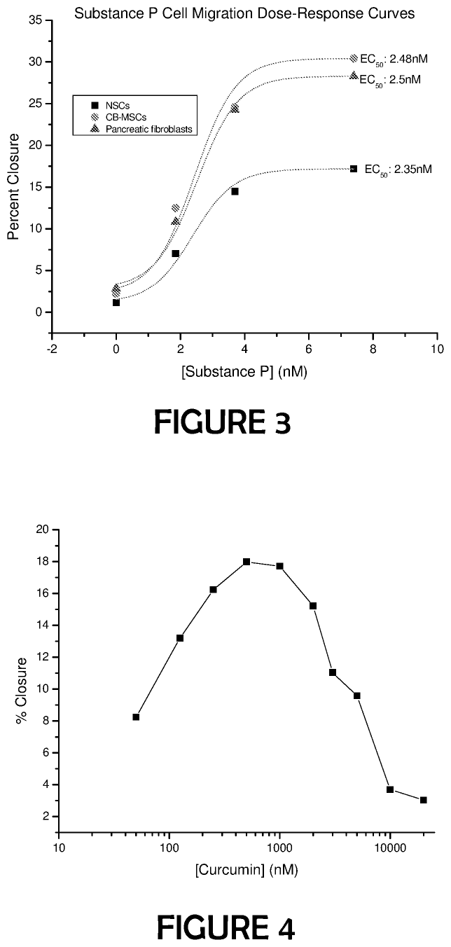

FIG. 3 is a line graph representation illustrating migration of various human cell lines exposed to Substance P. FIG. 3 shows the dose response relationship for Substance P-induced migration of MSCs, a primary human pancreatic cell line, and NSCs as well. The data shows the dose-response curve of MSCs (red circles), human primary pancreatic fibroblasts (blue triangles), and human NSCs (black squares) together with ECso values. Percent closure was determined at 24 hours.

By fitting the data to a sigmoidal curve, ECso values of 2.48, 2.5 and 2.35 nM were calculated for MSCs, human pancreatic fibroblasts, and NSCs, respectively. The EC₅₀ obtained for human pancreatic fibroblasts (i.e., 2.5 nM) compares well with a prior study of human fibroblast migration induced by Substance P of 2.2 nM using a suspension culture system to measure cell migration. Human fibroblast migration was mediated through the NK-1 receptor, since NK-1 receptor agonists mimicked Substance P and NK-1 receptor antagonists blocked Substance P induction of fibroblast migration. Fibroblast migration induced by Substance P is an important response to injury in addition to the induction of MSC migration. (Parenti, A., et al., Naunyn Schmiedeberg's Arch Pharmacol 353:475-481, 1996.) Since the EC50 for Substance P is comparable to prior results, these results provide validation support for the cell migration assay. The cell migration assays described above were set up as follows: One million cells/cell line were resuspended in 10 mL MSC-Gro™ serum free, quiescent medium (Vitro Biopharma Cat. No. SCO0B17) containing 5 μg/mL mitomycin C (Sigma, Cat. No. M4287) to inhibit proliferation and incubated for 2 hours at room temperature with end-to-end agitation at 7 RPM.

In some experiments, curcumin was used at 1 μM to block proliferation. Cells were centrifuged (450×g) for 7 minutes, washed with PBS and then resuspended in 1 mL MSC-Gro™ low serum, complete medium (Vitro Biopharma Cat. No. SCO0B1) and plated at 25,000 cells/well in black 96 well, TC-coated cell culture plates (ThermoScientific, Cat. No. 165305) containing cell seed-stoppers (Platypus, Cat. No. CMAUFL4) to form a cell free zone at the center of the well and incubated in 5% CO₂, 1% O₂, 94% N₂ at 37° C. in a humidified chamber for 24 hrs. Plates used for culture of NSCs were first treated with 10 μg/mL fibronectin (Sigma, Cat. No. F0556) for 2 hours at 37° C. Following washout with PBS (3×), cell seed stoppers were inserted, NSCs were plated at 25,000/well and incubated in 5% CO₂, 1% O₂, 94% N₂ at 37° C. in a humidified chamber for 48 hrs.

For studies of the effects of CXCR4 and MMP-9 inhibition following 24 hours of cell culture, appropriate wells were dosed with 15 μM GM6001 and 20 μM AMD3100 for 6 hours. Cells were washed once with PBS then incubated in serum free, MSC-Gro™ (Vitro Biopharma Cat. No. SCO0B17) containing 5 μM Cell Tracker Green CIVIFDA (Molecular Probes, Cat. No. C7025) at 37° C. for 30 minutes. The wells were then washed with serum free, MSC-Gro™ (Vitro Biopharma Cat. No. SCO0B17) and incubated for 30 minutes at 37° C. Cells were washed once with PBS and this was replaced with MSC-Gro™ serum free, quiescent medium (Vitro Biopharma Cat. No. SCO0B17) containing different concentrations of activating agents. Substance P was from Tocris Bioscience, (Cat. No. 1156) and curcumin from Santa Cruz Biotechnology (Cat. No. SC-200509A), VPA from Reagents Direct (Catalog Number 25-B43) and lithium chloride from Sigma Chemical Co. (Catalog number L4408). A TopSeal (PerkinElmer, Cat. No. 6050195) covered the plate for live-cell imaging in a BioTek Cytation3 imaging reader. Kinetic data was acquired every 2 hrs for 24 hrs using a GFP filter and bright field data acquisition. The gas phase throughout the acquisition of kinetic data was 5% O₂, 5% CO2 with the balance nitrogen maintained by a BioTek CO2/O2 gas controller. Images were saved as TIFF files to calculate percent closure using imaging data.

Example 3: Effect of curcumin on human MSCs/NSCs migration: Because curcumin had been previously shown to enhance rat NSC proliferation and enhanced recovery of spinal cord injury by use of NSCs pre-treated with curcumin (Ormond, D R, et al, PLoS ONE 9: e88916, 2014), the effects of curcumin were tested on MSC migration. The results are shown in FIG. 4, which is a line graph representation illustrating less than 1 μM curcumin induced migration of human MSCs and 1 to 10 μM curcumin blocked migration due to apparent toxicity. Percent closure was determined after a 36-hour run period. At concentrations less than 1 curcumin induced migration with an apparent EC₅₀ of 250 nM and at concentrations greater than 1 μM, curcumin blocked migration by apparent toxicity indicated by reduced cell number with an estimated LD₅₀ of 3 μM. These results also compare with prior studies (Ormond, D R, et al, PLoS ONE 9: e88916, 2014) providing further validation support for the cell migration assay. In addition to inducing proliferation of NSCs, curcumin also induces migration of MSCs and is toxic to MSCs at concentrations greater than 1 μM. Curcumin is thus emerging as a natural substance that serves as an activator of adult stem cells including MSCs and NSCs through increased stem cell proliferation and migration.

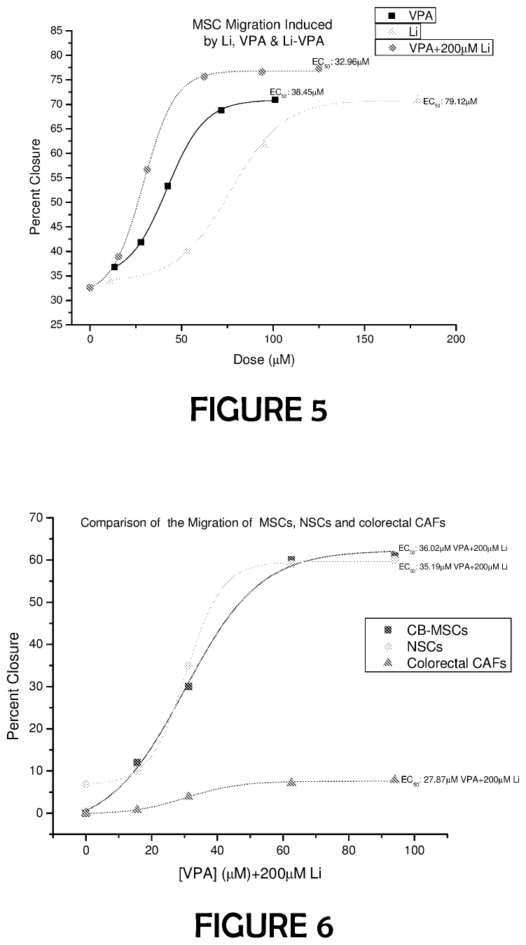

Example 4: Effects of lithium and VPA on migration of human stem cells: FIG. 5 is a line graph representation illustrating migration of MSCs induced by exposure to lithium alone (green triangles), VPA alone (black squares) and VPA in the presence of 200 μM lithium (red circles). Percent closure is plotted as a function of dose and the data was modeled by sigmoidal curve fitting to calculate EC₅₀ values. FIG. 5 shows lithium-induced MSC migration with a calculated EC₅₀ of 79.12 μM and maximal migration at 200 μM lithium. VPA also induced MSC migration with an EC₅₀ of 38.45 μM with maximum closure at 100 μM. Since maximal lithium-induced closure occurred at 200 μM, the closure induced by increasing VPA concentrations in 200 μM lithium was then investigated. The results showed a lower EC₅₀ than that observed with VPA only (i.e., 32.96 μM) suggesting a synergistic effect of Li-VPA on MSC migration.

The effect of Li-VPA on cell migration of different cell lines was determined; the results are shown in FIG. 6, which is a line graph representation illustrating migration of CB-MSCs, NSCs and colorectal CAFs by induced by increasing VPA concentration in 200 μM lithium. Percent closure is plotted as a function of VPA in medium containing 200 μM lithium. Percent closure is plotted as a function of dose and the data was modeled by sigmoidal curve fitting to calculate EC₅₀ values. Migration of MSCs (blue squares), NSCs (green circles) and colorectal CAFs (red triangles) are shown together with calculated EC₅₀ values. While increasing VPA in 200 μM lithium showed similar kinetics between MSCs and NSCs, EC₅₀ was 36.02 and 35.19 μM, colorectal CAFs did not migrate to the same extent as MSCs and NSCs and similar data was obtained for other CAFs (data not shown). Thus, while MSCs and NSCs are robustly induced to migrate by Li-VPA, CAFs do not similarly migrate.

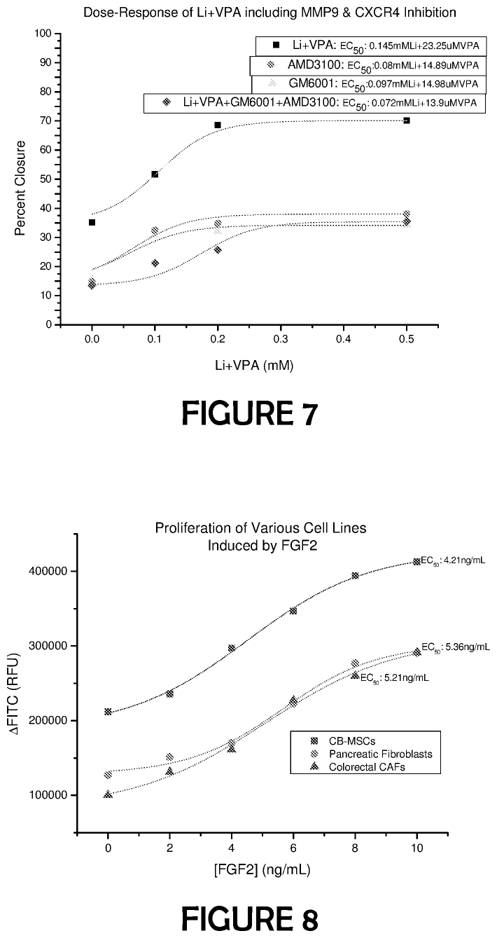

The molecular mechanisms of the effect of lithium and VPA were then investigated on stem cell migration. Since a prior report suggested that VPA up-regulated CXCR4, a critical chemokine receptor involved with cellular mobility, and that lithium up-regulated MMP-9 (Tsai, L K, et al., Stroke 42(10): 2932-2939, 2011), the effects of known inhibitors of CXCR4 and MMP-9 on the migration of MSCs were determined. The results are shown in FIG. 7, which is a line graph representation illustrating migration of human MSCs exposed to a combination of lithium and VPA with and without inhibition of MMP9 and CXCR4. The data shows the response curve of MSCs treated with Li and VPA alone (black squares), treated with Li and VPA and with inhibition of CXCR4 by AMD3100 (red circles), treated with Li and VPA and inhibition of MMP9 by GM6001 (green triangles), and treated with Li and VPA while inhibiting both CXCR4 and MMP9 (blue diamonds). Percent closure is plotted as a function of dose. The results indicate that the CXCR-4 inhibitor, AMD 3100, blocked the VPA-induced CB-MSC migration and that GM 6001, a competitive inhibitor of MMP-9, blocked lithium induced CB-MSCs. Also, in the presence of both AMD 3100 and GM6001, MSC migration induced by Li-VPA was also blocked. These results suggest that CXCR4 and MMP-9 are molecular components of the VPA and lithium-induced migration of MSCs thus confirming and extending prior results of Tsai, L K, et al., Stroke 42:2932-2939, 2011.

Example 5: Effects of lithium and VPA on stem cell proliferation: The effects of lithium and VPA on stem cell proliferation were then investigated using an assay based on Presto Blue, a fluorescent marker of cellular reduction. FIG. 8 is a line graph representation illustrating proliferation of different cell lines induced by increasing concentrations of FGF-b (FGF-2) after a 5 day exposure. Relative fluorescent units were measured using a FITC filter on a Modulus microplate reader and plotted as a function of FGF-2. The data was modeled by sigmoidal curve fitting to calculate EC₅₀ values. FIG. 8 shows the effect of FGF-2 on the proliferation of different cell lines including CAFs, primary fibroblasts and MSCs. Proliferation of these mesenchymal cells was stimulated by recombinant human FGF-2 with EC₅₀ values in the range of 3 to 6 ng/ml with maximal proliferative responses at about 10 ng/ml. Since these results are comparable to previous studies (Lee, T H, et al, Biochem Cell Biol. 26: 1-8, 2015), these data provide validation support for the cell-based proliferation assay.

The proliferative effects of lithium and VPA were compared, either alone or in combination to MSC proliferation. The results are shown in FIG. 9, which is a line graph representation illustrating proliferation of MSCs induced by lithium, VPA and VPA in 200 μM lithium after a 5 day exposure. Relative fluorescent units were measured using a FITC filter on a Modulus microplate reader and plotted as a function of FGF-2. The data was modeled by sigmoidal curve fitting to calculate EC₅₀ values. The EC₅₀ value was 76.7 μM for lithium. This was comparable to the lithium effect on MSC migration (FIG. 5). The EC₅₀ for VPA was 47.71 μM. In the presence of 200 μM lithium, the EC₅₀ for VPA was 63.31 μM. While MSC migration showed an apparent synergy with combined Li-VPA, this does not appear to be the case for MSC proliferation, since larger VPA dosages were needed for equivalent MSC proliferation in the presence of 200 μM lithium.

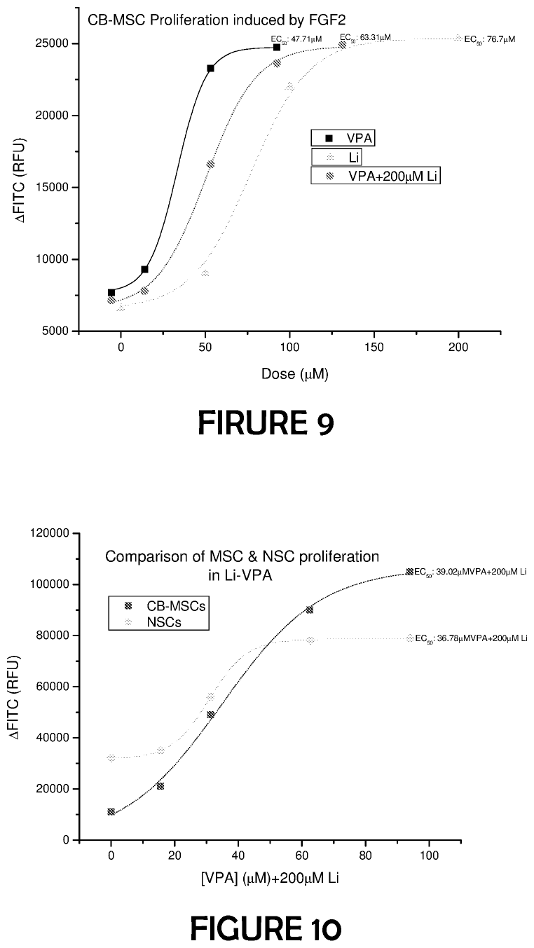

The proliferative responses of MSCs and NSCs were compared to Li-VPA. The results are shown in FIG. 10, which is a line graph representation illustrating proliferation of MSCs and NSCs as a function of increasing VPA in 200 μM lithium after a 5 day exposure. Relative fluorescent units were measured using a FITC filter on a Modulus microplate reader and plotted as a function of FGF-2. The data was modeled by sigmoidal curve fitting to calculate EC₅₀ values. While both NSCs and MSCs exhibited comparable EC₅₀, 36.78 and 39.02 μM, respectively, the extent of proliferation is reduced in NSCs compared to MSCs. This may reflect intrinsic proliferative capacity. Also, since the cell migration results yielded similar results, both proliferation and migration of MSCs and NSCs may be activated at similar reduced dosage when lithium and VPA are administered in combination. Thus, in the presence of 200 μM lithium, VPA is 50% effective in inducing cell proliferation and migration at 35 to 39 μM (mean 37 μM).

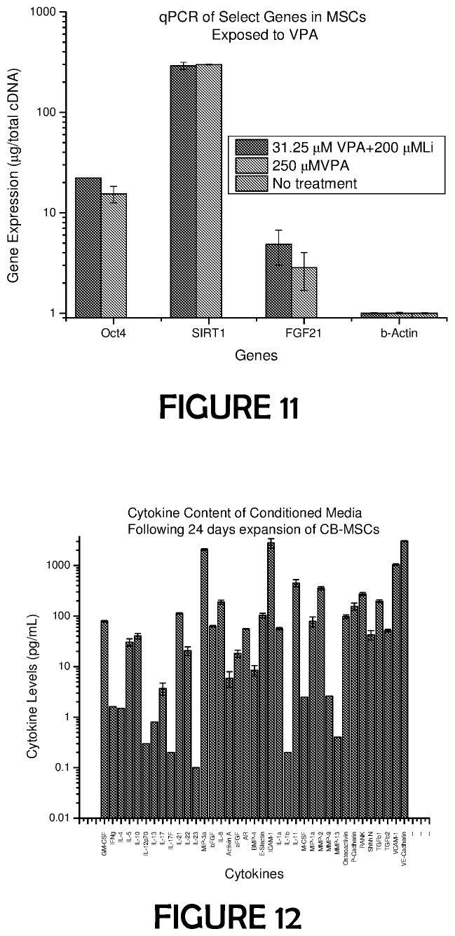

Example 6: Effects of VPA and lithium on stem cell gene expression: In FIG. 11, results of gene expression analysis are shown following the exposure of MSCs to VPA. FIG. 11 is a bar graph representation illustrating PCR used to measure target genes known to be subject to epigenetic regulation by HDAC inhibitors. The graph shows the result of human MSCs treated with VPA alone or in combination with lithium. The expression of Oct 3/4, a well known pluripotency gene, was increased about 20-fold compared to untreated human MSCs. The expression of SIRT-1 was highly elevated compared to untreated MSC by—300-fold without differences between treatment with either VPA or lithium-VPA. The expression of FGF-21 was also elevated by 3- to 5-fold and its expression was higher in MSCs treated with Li-VPA, although this increase with not significant. All gene expression was normalized to untreated MSCs.

Beta actin was measured as a house-keeping gene. The FIG. 11 graph shows the result of gene expression analysis of human MSCs treated with VPA only or VPA+ lithium. Gene expression was quantified by determining the amount of gene-specific DNA/total cDNA. Data is mean+/−SD of 4 replicates. These results thus show increased expression of Oct 3/4, SIRT-1 and FGF-21 as a result of exposure to VPA or Li-VPA using 200 μM lithium and 31.25 μM VPA.

Native human umbilical cord-derived MSCs (Vitro Biopharma Cat. No. SC00A1) were expanded from cryopreservation in T-25 TC-coated flasks (BD Falcon, Cat. No. 353108) in MSC-Gro™ low serum, complete medium (Vitro Biopharma Cat. No. SCO0B1). Cells were sub-cultured and counted on a Beckerman-Coulter Z2 particle counter (range 10 μm-30 μm). Cells were plated at 10,000/cm² a TC-coated Greiner Bio-One T75 flask and maintained in MSC-Gro™ serum free, complete medium (Vitro Biopharma Cat. No. SCO0B3) in a reduced O₂ environment (1% O₂, 5% CO₂, 94% N₂) at 37° C. in a humidified chamber. The MSCs were treated continuously for up to 2 weeks. Cultures were fed every 3 days. Cells were harvested using Accutase (Innovative Cell Technologies Inc., Cat No AT-104) and centrifuged (450×g) for 7 minutes. Cell supernatant was aspirated off and cells were resuspended in 1 mL PBS and counted on a Beckerman-Coulter Z2 particle counter (range 10 μm-30 μm). Total RNA was extracted using RNeasy mini kit (Qiagen Cat. No. 74104). RNA was quantified using an absorbance measurement at 260 nm. RNA was converted to cDNA using Quantitect reverse transcription kit (Qiagen Cat. No. 205310) in a thermocycler. RNA was incubated in the gDNA elimination reaction for 2 minutes at 42° C. then incubated in the reverse-transcription master mix for 15 minutes at 42° C. Immediately after, it was incubated at 95° C. for 3 minutes for inactivation. cDNA was sent to an outside lab (CU-Anschutz Metabolic Laboratory) for q-PCR to detect relative or absolute gene expression levels. cDNA was diluted 1:5 and an iTaq Universal Supermix fluorescent probe (BioRad Cat. No. 172-5120) used to detect the threshold cycle (Ct) during PCR Dilution factors and cDNA concentrations were calculated into recorded values then normalized to untreated hMSCs (Vitro Biopharma Cat. No. SC00A1).

Example 7: Secretion of cytokines from MSCs: Preliminary experiments were performed concerning the composition of cytokines within the soluble factors secreted from stem cells and the results are shown in FIG. 12, which is a bar graph representation illustrating cytokine levels in cell culture media exposed to MSCs for 24 days and determined by microarray analysis. Conditioned media was analyzed using an inflammation Th17 microarray and a bone metabolism array. Results show an increase in inflammatory cytokines as well as adhesion factors. With a substantial increase in the content of MIP-3a, ICAM-1, IVCAM-1 and VE-Cadherin within the conditioned medium, this suggests the well known immunosuppressive role of MSCs.

Native human umbilical cord-derived MSCs (Vitro Biopharma Cat. No. SC00A1) were plated at 1,000/well in a tissue cultured 6-well plate (BD Falcon, Cat Number 353046) in Gro™ low serum, complete medium (Vitro Biopharma Cat. No. SCO0B1). Cells were continuously grown for a period of 30 days. Conditioned media was collected at day 3, 6, 12, 18, and 24. Multiple microarrays were run using conditioned media for cytokine secretion determination after a continuous 24 day culture period. An inflammation microarray (Ray Biotech, Cat. No. QAH-INF-3), a bone metabolism microarray (Ray Biotech, Cat. No. QAH-BMA-1) and a Th17 microarray (Cat. No. QH-TH17-1) were used to analyze the conditioned media. A laser scanner (Molecular Probes, Genepix 4000B) was used to measure the fluorescent signals of each microarray.

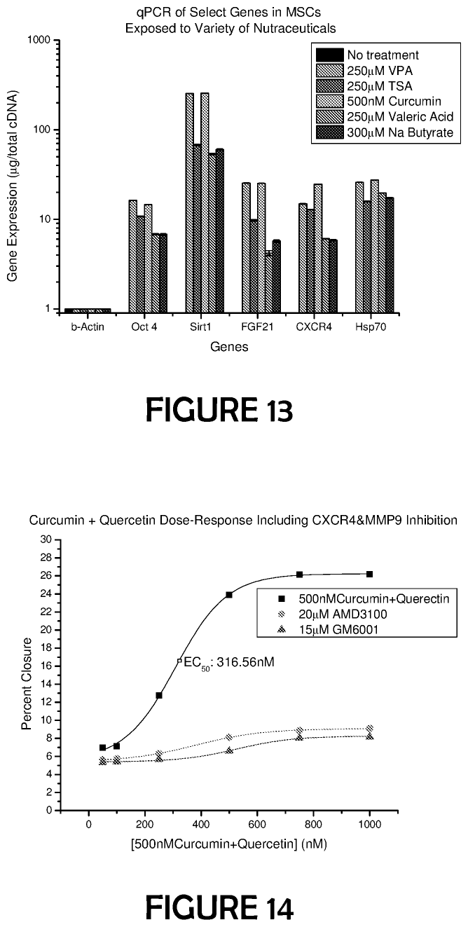

Example 8: Effects of nutraceuticals on stem cell gene expression: In FIG. 13, results of gene expression analysis are shown following the exposure of MSCs to VPA and various nutraceuticals known to exhibit epigenetic effects. The expression of various target genes as determined by qPCR is shown as compared to the house-keeping gene, (β-actin. The expression of Oct 3/4, a well known stem cell pluripotency gene, was increased about 20-fold compared to β-actin in human MSCs by VPA and curcumin while TSA, valeric acid and Na butyrate resulted in less expression activation. The expression of SIRT-1 was highly elevated by—300-fold without differences between treatment with either VPA or curcumin while TSA, valeric acid and Na butyrate were significantly less effective. Similar yet lower expression levels of FGF-21, CXCR4 and Hsp70 were observed (maximum of 25-fold) with VPA and curcumin showing nearly equivalent expression increases and lower levels with TSA, valeric acid and Na butyrate. All gene expression was normalized to that of untreated MSCs. Beta-actin was used as a house-keeping gene.

Gene expression was quantified by determining the amount of gene-specific DNA/total cDNA. Data is mean+/−SD of 4 replicates. These results thus show highest increased expression of Oct 3/4, SIRT-1, FGF-21, CXCR4, and Hsp70 as a result of exposure to 250 μM VPA and 500 nM curcumin, the latter possibly due to its HDAC inhibitory effects (Soflaei S S, et. al, Curr Pharm Des. 2018; 24(2):123-129 although it has other epigenetic effects as well. Regardless of the precise mechanisms, curcumin showed equivalent or increased expression of Oct 3/4, SIRT-1, FGF21, CXCR4 and Hsp70 as compared to VPA.

Native human umbilical cord-derived MSCs (Vitro Biopharma Cat. No. SC00A1) were expanded from cryopreservation in T-25 TC-coated flasks (BD Falcon, Cat. No. 353108) in MSC-Gro™ low serum, complete medium (Vitro Biopharma Cat. No. SCO0B1). Cells were sub-cultured and counted on a Beckerman-Coulter Z2 particle counter (range 10 μm-30 μm). Cells were plated at 10,000/cm2 a TC-coated Greiner Bio-One T75 flask and maintained in MSC-Gro™ serum free, complete medium (Vitro Biopharma Cat. No. SCO0B3) in a reduced O2 environment (1% O₂, 5% CO₂, 94% N₂) at 37° C. in a humidified chamber. The MSCs were treated continuously for up to 2 weeks. Cultures were fed every 3 days. Cells were harvested using Accutase (Innovative Cell Technologies Inc., Cat No AT-104) and centrifuged (450×g) for 7 minutes. Cell supernatant was aspirated off and cells were resuspended in 1 mL PBS and counted on a Beckerman-Coulter Z2 particle counter (range 10 μm-30 μm).

Total RNA was extracted using RNeasy Mini Kit (Qiagen Cat. No. 74104). RNA was quantified using an absorbance measurement at 260 nm. RNA was converted to cDNA using Quantitect reverse transcription Kit (Qiagen Cat. No. 205310) in a thermocycler. RNA was incubated in the DNA elimination reaction for 2 minutes at 42° C. then incubated in the reverse-transcription master mix for 15 minutes at 42° C. Immediately after, it was incubated at 95° C. for 3 minutes for inactivation. cDNA was sent to an outside lab (CU-Anschutz Metabolic Laboratory) for q-PCR to detect relative or absolute gene expression levels. cDNA was diluted 1:5 and an iTaq Universal Supermix fluorescent probe (BioRad Cat. No. 172-5120) was used to detect the threshold cycle (Ct) during PCR. Dilution factors and cDNA concentrations were calculated into recorded values then normalized to untreated hMSCs (Vitro Biopharma Cat. No. SC00A1).

Example 9: Effects of nutraceuticals on stem cell migration. FIG. 14 shows the migration of MSCs induced by exposure to 500 nM curcumin and variable concentrations of quercetin (black squares) alone, or in the presence of the CXCR4 inhibitor (AMD3100) (red circles) and the MMP9 inhibitor (GM6001) (blue triangles). Percent closure is plotted as a function of dose and the data was modeled by sigmoidal curve fitting to calculate EC₅₀ values. FIG. 14 shows curcumin and quercetin induced MSC migration with a calculated EC₅₀ of 316.56 nM quercetin and maximal migration of the combination of curcumin and quercetin. The CXCR4 inhibitor, AMD3100, blocked MSC migration as did the MMP9 inhibitor, GM6001, suggesting a similar molecular mechanism for the migration of MSCs by curcumin and quercetin as for stem cell migration induced by VPA-Li.

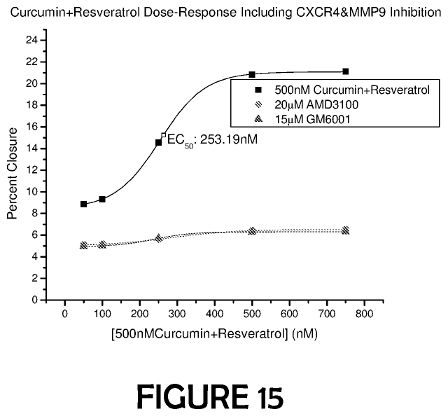

FIG. 15 shows the migration of MSCs induced by exposure to 500 nM curcumin and variable concentrations of resveratrol (black squares) alone, or in the presence of the CXCR4 inhibitor (AMD3100) (red circles) and the MMP9 inhibitor (GM6001) (blue triangles). Percent closure is plotted as a function of resveratrol dose (nM) and the data was modeled by sigmoidal curve fitting to calculate EC₅₀ values. These results show that curcumin and resveratrol-induced MSC migration with a calculated EC₅₀ of 253.19 nM resveratrol and maximal migration of the combination of curcumin and resveratrol. The CXCR4 inhibitor, AMD3100, blocked MSC migration as did the MMP9 inhibitor, GM6001 suggesting a similar molecular mechanism for the migration of MSCs by curcumin and resveratrol as for stem cell migration induced by Li.

Example 10: Extraction of Mononuclear Cells from Human Umbilical Cord:

Methods: Human umbilical cord was obtained from CryoPoint, Brownsville, Ind. under informed consent from the donor. It was transported in saline and maintained at 4° C. during transport. The umbilical cord was micro-dissected into 1-2 cm² pieces in a petri dish containing phosphate buffered saline (PBS) within a biological safety cabinet. All subsequent procedures occurred in a sterile environment. An enzymatic digestion mixture was prepared in 0.2 PZ U/ml Collagenase NB 6 GIVIP grade, Serva Chemicals, in HEPES-buffered saline at 2.5 ml/gm umbilical cord tissue. This mixture was incubated at 37° C. in an end-over-end rotator at 25 RPM. Following incubation, cells and tissue were separated using a Buchner funnel (90 to 130-micron pore size) following washout of residual cells, with 3×20 ml PBS. Cells were pelleted by centrifugation at 440×G for 15 minutes and re-suspended in 10-20 ml PBS for cell counting including total cell count (10 to 40 micron) and acridine-orange, propidium iodide using the Countess II instrument (Fisher Scientific). The mononuclear cells were re-suspended at 10⁶ MNC/ml in CryoStore 2 cryopreservation medium (BioLife Solutions, Catalog Number 202102) and cryopreserved using controlled rate freezing from room temperature to −80° C. at ˜1° C./minute, followed by storage in liquid nitrogen.

Results: The above procedures yielded a total cell count of 530 million, consisting of 13 million MNCs at 47.3% viability.

### Expansion of MSCs

Methods: Pass 0 cultures (i.e., initial passage following collagenase digest) were cultured at 15K to 20K total cells/cm2 in T-75 TC-coated tissue culture flasks (Falcon, Catalog Number 353136) in MSC cell culture medium (MSC-Gro™, Vitro Biopharma catalog number SCO0B4-3, clinical grade humanized, serum-free medium (supplemented to 5% Human serum AB, Golden West Biologicals, Temecula, Calif.) plus 1× penicillin/streptomycin (Sigma, catalog number P4383). The cultures were maintained in a tri-gas incubator (HerraCell, 240i; Fisher Scientific) set to 5% O₂ and 5% CO₂. These cultures were monitored at 4- to 5-day intervals for colony formation and expansion. Pass 0 cultures were maintained for 10 to 14 days with feeding at every 3 days following establishment of cultures at about 7 days. At 80% to 90% confluence, the Pass 0 cultures were sub-cultured using TrypLE™ Select (1×) (Gibco, Catalog Number 12563-029) using 30-minute incubation at 37° C. with agitation at 75 RPM. Similar procedures were used in Passes 1, 2, and 3, except that T-1000 flasks were used for these passages and appropriate scale-up in media volumes used for culture, TrypLE™ Select, etc.

Results: A single T-75 culture of pass 0 cells yielded on average 8 to 10 million MSCs at >85% viability. Subsequent passages yielded 10 to 12-fold increases in the number of inoculated MSCs at greater than 90% viability.

The genotype was found to be:

| | | | | | |
|---|---|---|---|---|---|
| Arneloge0.1 | X | Y | D18S51 | 16 | 17 |
| vWA | 17 | 18 | Penta E | 14 | 20 |
| D8S1179 | 14 | | D5S818 | 13 | |
| TPOX | 8 | 9 | D13S317 | 9 | 13 |
| FGA | 19 | 24 | D7S820 | 10 | 12 |
| D3S1358 | 16 | 17 | D16S539 | 10 | 13 |
| THO1 | 7 | 9 | CSF1PO | 10 | 11 |
| D21S11 | 30 | 31.2 | Penta D | 11 | 12 |

Since other modifications and changes varied to fit particular operating requirements and environments will be apparent to those skilled in the art, this invention is not considered limited to the example chosen for purposes of this disclosure, and covers all changes and modifications which does not constitute departures from the true spirit and scope of this invention.

Having thus described the invention, what is desired to be protected by Letters Patent is presented in the subsequently appended claims:

1. A method of treating a medical condition by stem cell therapy, the steps comprising:
a) transplanting stem cells into or onto a patient having the medical condition or suspected of having the medical condition; and

2. increasing stem cell proliferation, activating migration to sites of inflammation and epigenetic effects, regenerating damaged tissues through inhibition of apoptosis, inhibition of inflammation and oxidative stress, and inducing angiogenesis or stem cell differentiation into various cells by administering substances to the patient that induce GSK-3 beta inhibition and HDAC inhibition.

3. The method of claim 1, wherein the stem cells comprise mesenchymal stem cells derived from the patient receiving the transplant.

4. The method of claim 1, wherein the stem cells comprise mesenchymal stem cells derived from donor tissues, wherein the donor tissues are not from the patient receiving the transplant.

5. The method of claim 1, wherein the stem cells comprise neural stem cells.

6. The method of claim 1, wherein the medical condition is selected from the group consisting of: ARD secondary to viral infection, osteoarthritis, tendonitis, herniated disc, ligament damage, acute and chronic kidney disease and injury, Parkinson's disease, traumatic brain injury, Alzheimer's disease, amyotrophic lateral sclerosis, spinal cord injury, other skeletal muscular disorders, Crohn's disease, cardiovascular disease, Graft-versus host disease, liver dysfunction or diseases, Type 1 diabetes, Type 2 diabetes, myocardial ischemia, heart failure, coronary artery disease, and other disorders characterized by inflammation.

7. A method of treating medical conditions by stem cell therapy, the steps comprising:
a) transplanting stem cells into and onto the patient having the medical condition or suspected of having the medical condition; and
b) increasing expression of Sirt-1 by at least 200-fold, increasing expression of CXCR4 by at least 10-fold; increasing heat shock protein 70 by at least 20-fold, increasing expression of Oct 3/4 by at least 10-fold, increasing expression of FGF21 by at least 20-fold, increasing proliferation and migration of human mesenchymal stem cells, and inducing stem cell migration through increased activation of CXCR-4 and MMP-9 by administering substances to the patient that induce GSK-3 beta inhibition and HDAC-1 inhibition.

8. The method of claim 6, wherein the stem cell transplant comprises MSCs derived from a patient receiving the transplant.

9. The method of claim 6, wherein the stem cell transplant comprises MSCs derived from donor tissues, wherein the donor tissues are not from the patient receiving the transplant.

10. The method of claim 6, wherein the stem cell transplant comprises stem cell-derived neurons.

11. The method of claim 6, the steps further comprising:
c) treating medical conditions selected from the groups consisting of: ARDS secondary to viral infection, osteoarthritis, tendonitis, herniated disc, ligament damage, acute and chronic kidney disease and injury, Parkinson's disease, traumatic brain injury, Alzheimer's disease, amyotrophic lateral sclerosis, spinal cord injury, other skeletal muscular disorders, Crohn's disease, cardiovascular disease, Graft-versus host disease, liver dysfunction or diseases, Type 1 diabetes, Type 2 diabetes, myocardial ischemia, heart failure, coronary artery disease, and other disorders characterized by inflammation.
